(19) United States
(12) Patent Application Publication
(10) Pub. No.: US 2010/0210959 A1
(43) Pub. Date: Aug. 19, 2010

(54) PORTABLE ELECTROCARDIOGRAPH SET

(75) Inventor: Hiromichi Karo, Kyoto (JP)

Correspondence Address: RABIN & Berdo, PC, 1101 14TH STREET, NW, SUITE 500, WASHINGTON, DC, 20005, US

(73) Assignee: Omron Healthcare Co., Ltd., Kyoto (JP)

(21) Appl. No.: 12/678,689

(22) PCT Filed: Jun. 12, 2008
(86) PCT No.: PCT/JP2008/060765

§ 371 Date: Mar. 17, 2010

(30) Foreign Application Priority Data
Sep. 28, 2007 (JP) 2007-255074

(51) Int. Cl. A61B 5/0404

(52) U.S. Cl. 600/509

(57) ABSTRACT

A portable electrocardiograph set includes an electrocardiograph body provided with an electrocardiogram monitoring circuit, a lead part having two or more cables with respective clips for connecting external electrodes to the electrocardiogram monitoring circuit, and a containing member for containing and carrying the electrocardiograph body and the lead part. The clip fixing member is configured to enable all of the clips of the two or more cables simultaneously fixed thereto. The clip fixing member may be provided attached or not attached to the containing member, the lead part or the electrocardiograph body.

### BACKGROUND

The invention is related to a portable electrocardiograph set, more specifically a portable electrocardiograph set provided with an electrocardiograph body, a lead part having cables with clips connected to external electrodes and a containing member for containing and carrying the electrocardiograph body and the lead part.

Generally, patients' cardiograms are used to diagnose ischemia diseases such as arrhythmia, angina, and cardiac infarct. Various types of electrocardiographs with different configurations are known and used in order to obtain such electrocardiograms. One of such electrocardiographs is known as a portable electrocardiograph, which can be carried by a subject to monitor and store the electrocardiographic waveform when a subjective symptom occurs, by the subject contacting the electrodes to his or her own body.

A portable electrocardiograph is known having exposed electrodes which are contacted to the subject's body such that the subject himself or herself can quickly monitor the electrocardiographic waveform.

A portable electrocardiograph is also known having external electrodes extending from the electrocardiograph body through cables such that a person other than the subject (for example, a medical doctor or nurse) can contact the extended electrodes to the subject's body in order to monitor the electrocardiographic waveform of the subject.

Disposable external electrodes are often used especially for the latter type of portable electrocardiograph. On such occasions, the portable electrocardiograph body is usually accompanied by cables with clips to enable electrical contact between the electrocardiogram monitoring circuit provided in the electrocardiograph body and the external electrodes.

### BRIEF SUMMARY

A portable electrocardiograph set according to an embodiment of the invention includes an electrocardiograph body provided with an electrocardiogram monitoring circuit, a lead part having two or more cables with respective clips for connecting external electrodes to the electrocardiogram monitoring circuit, and a containing member for containing and carrying the electrocardiograph body and the lead part. The clip fixing member is configured to enable all of the clips of the two or more cables simultaneously fixed thereto. The clip fixing member is provided at either the containing member, the lead part or the electrocardiograph body.

A portable electrocardiograph set according to an embodiment of the invention includes an electrocardiograph body provided with an electrocardiogram monitoring circuit, a lead part having two or more cables with respective clips for connecting external electrodes to the electrocardiogram monitoring circuit, a clip fixing member configured to enable all of the clips included in the two or more cables simultaneously fixed thereto, and a containing member for containing and carrying the electrocardiograph body, the lead part and the clip fixing member. The containing member is configured to contain the clip fixing member together with the lead part with the clips being fixed to the clip fixing member.

### BRIEF DESCRIPTION OF THE DRAWINGS

FIG. 1 is a perspective view showing the appearance structure of the electrocardiograph body included in the portable electrocardiograph set according to a first embodiment of the invention.

FIG. 2 is a perspective view showing the structural appearance of the electrocardiograph body included in the portable electrocardiograph set according to a first embodiment of the invention.

FIG. 3 is a front view of the electrocardiograph body shown in FIGS. 1 and 2.

FIG. 4 is a top view of the electrocardiograph body shown in FIGS. 1 and 2.

FIG. 5 is a bottom view of the electrocardiograph body shown in FIGS. 1 and 2.

FIG. 6 is a right side view of the electrocardiograph body shown in FIGS. 1 and 2.

FIG. 7 is a left side view of the electrocardiograph body shown in FIGS. 1 and 2.

FIG. 8 is a frame format showing the structural appearance of the lead part for connecting an external electrode contained in the portable electrocardiograph set according to the first embodiment of the invention.

FIG. 9 is a top view of the electrocardiograph body keeping open an access cover for inserting the lead part for connecting the external electrode or

FIG. 10 is a frame format showing either the lead part for connecting the external electrode or the lead part for connecting the external terminal being inserted into the electrocardiograph body.

FIG. 11 is a front view showing the structural appearance of a drawstring bag included as container member in the portable electrocardiograph set according to the first embodiment of the invention.

FIG. 12 is a function block diagram of the electrocardiograph body, etc. included in the portable electrocardiograph set according to the first embodiment of the invention.

FIG. 13 is a flowchart of the electrocardiograph body included in the portable electrocardiograph set according to the first embodiment of the invention.

FIG. 14 is a display example of the monitored results on the display of the electrocardiograph body included in the portable electrocardiograph set according to the first embodiment of the invention.

FIG. 15 is a display example of the monitored results on the display of the electrocardiograph body included in the portable electrocardiograph set according to the first embodiment of the invention.

FIG. 16 is a view showing a posture a subject should take when monitoring an electrocardiographic waveform in the portable electrocardiograph set according to the first embodiment of the invention.

FIG. 17 is a view showing a posture a subject should take when monitoring an electrocardiographic waveform in the portable electrocardiograph set according to the first embodiment of the invention.

FIG. 18 is a view showing the right hand of a subject holding an electrocardiograph body when the subject takes the postures shown in FIGS. 16 and 17.

FIG. 19 is a view showing an example of locations where the external electrodes should be attached when monitoring the electrocardiographic waveform by the second embodiment in the portable electrocardiograph set according to the first embodiment of the invention.

FIG. 20 is a view showing another example of locations where the external electrodes should be attached when monitoring the electrocardiographic waveform by the second embodiment in the portable electrocardiograph set according to the first embodiment of the invention.

FIG. 21 is a view showing a manner that the electrocardiograph body and the lead part for connecting external electrodes are contained in the drawstring bag as container member in the portable electrocardiograph set according to the first embodiment of the invention.

FIG. 22 is a view showing a state where the electrocardiograph body and the lead part for connecting external electrodes are contained in the drawstring bag as container member in the portable electrocardiograph set according to the first embodiment of the invention.

FIG. 23 is a frame format showing an electrical connection when a check for wire breakage is conducted in the portable electrocardiograph set according to the first embodiment of the invention.

FIG. 24 is a view showing a display example when no wire breakage occurs in the cables of the lead part for connecting external electrodes.

FIG. 25 is a view showing a display example when a wire breakage occurs in the cables of the lead part for connecting external electrodes.

FIG. 26 is a view showing a manner that the electrocardiograph body and the lead part for connecting external electrodes are contained in the drawstring bag as container member in the portable electrocardiograph set according to the second embodiment of the invention.

FIG. 27 is a view showing a manner that the electrocardiograph body and the lead part for connecting external electrodes are contained in the drawstring bag as container member in the portable electrocardiograph set according to the third embodiment of the invention.

### DETAILED DESCRIPTION

Hereinafter, an embodiment of the invention is described with reference to the drawings. The portable electrocardiograph set according to this embodiment is configured to monitor the electrocardiographic waveform based on a so-called three-point induction method. It is conveniently configured such that the waveforms may be monitored not only by using exposed electrodes provided on the surface of the electrocardiograph body, but also by using external electrodes. Therefore, the portable electrocardiograph set according to this embodiment described hereinafter is provided with not only the electrocardiograph body but the lead part for connecting external electrodes and a containing member for containing the electrocardiograph body and the lead part.

### First Embodiment

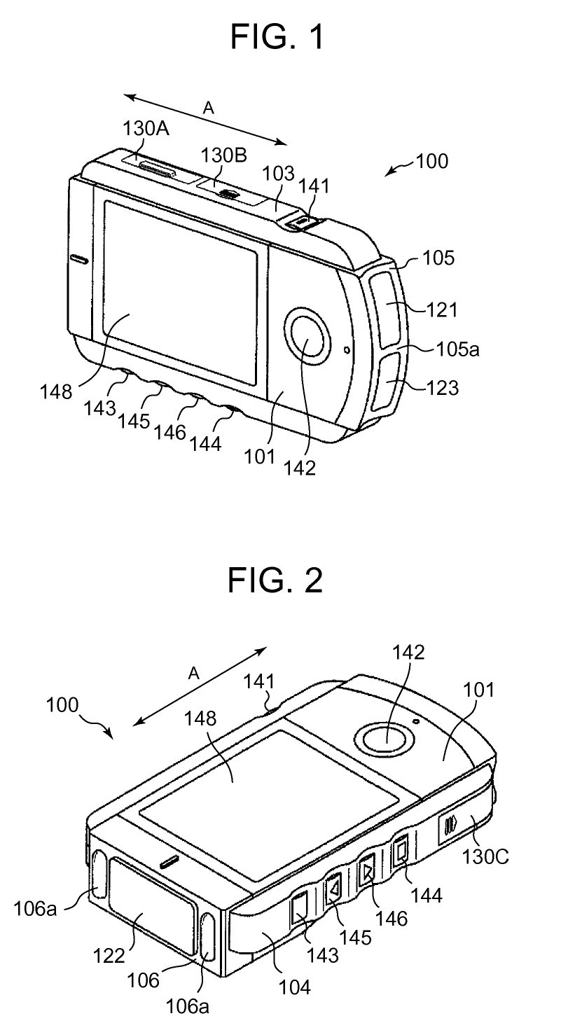

FIGS. 1 and 2 are perspective views showing the structure of the electrocardiograph body included in the portable electrocardiograph set according to the first embodiment of the invention.

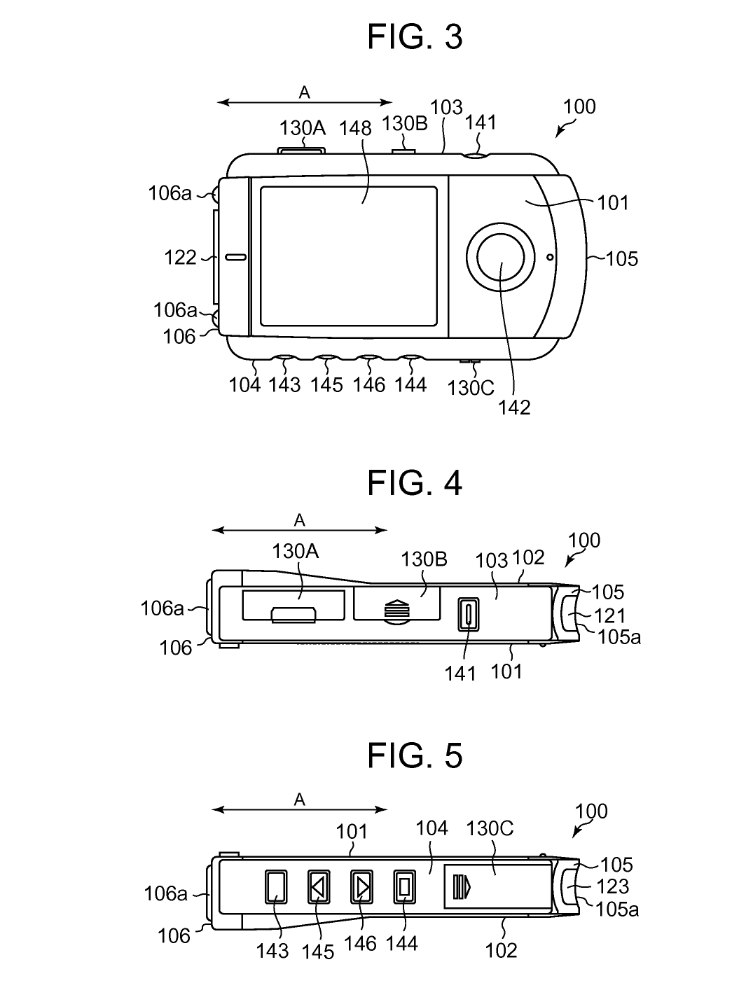

FIGS. 3 to 7 are a front view, top view, bottom view, right side view, and left side view of the electrocardiograph body shown in FIGS. 1 and 2, respectively. The outer appearance of the electrocardiograph body included in the portable electrocardiograph set according to this embodiment is described with reference to FIGS. 1 to 7.

The electrocardiograph body 100 included in the portable electrocardiograph set 1A according to this embodiment is configured to be small and light such that it can be held in one hand and conveniently carried. The electrocardiograph body 100 has a flat and elongated substantially rectangular shape and includes the after-mentioned display part 148, operating part 140, and exposed electrodes (negative electrode 121, positive electrode 122, and indifferent electrode 123), etc on the surface (front face 101, back face 102, top face 103, bottom face 104, right lateral face 105 and left lateral face 106).

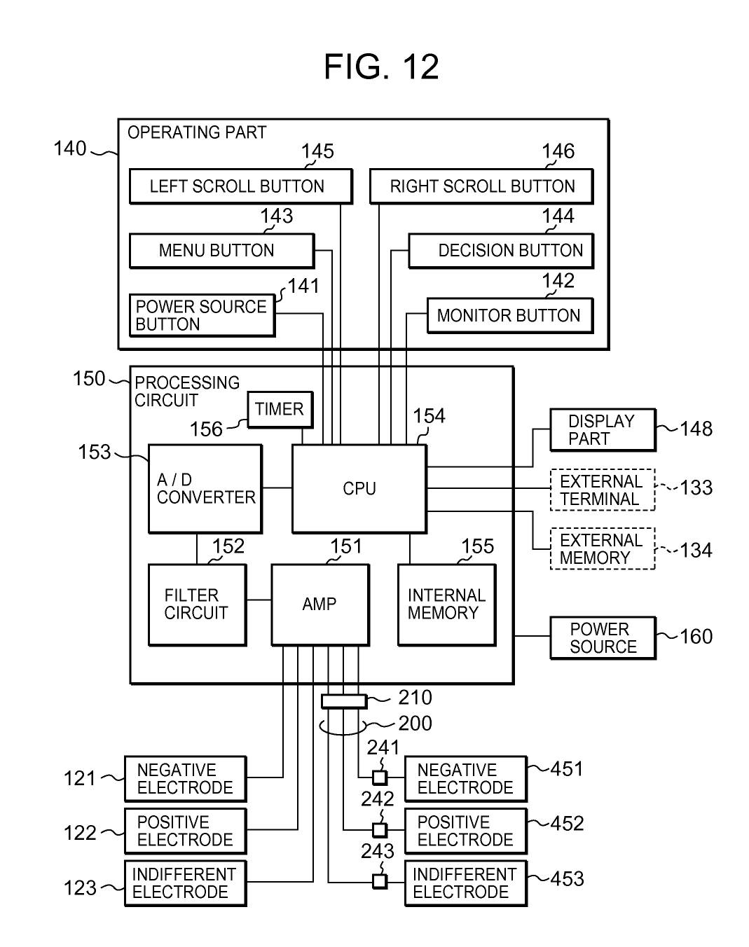

As shown in FIGS. 1 to 3, a monitor button 142 is provided on one end of a front face 101 in a longitudinal direction (in a direction of arrow A) of the electrocardiograph body 100. A display part 148 is provided on the other end. The monitor button 142 is included in the operating part 140 (see FIG. 12) to instruct a start of monitoring. The display part 148 is made up of, for example, a LCD which displays monitor results, etc.

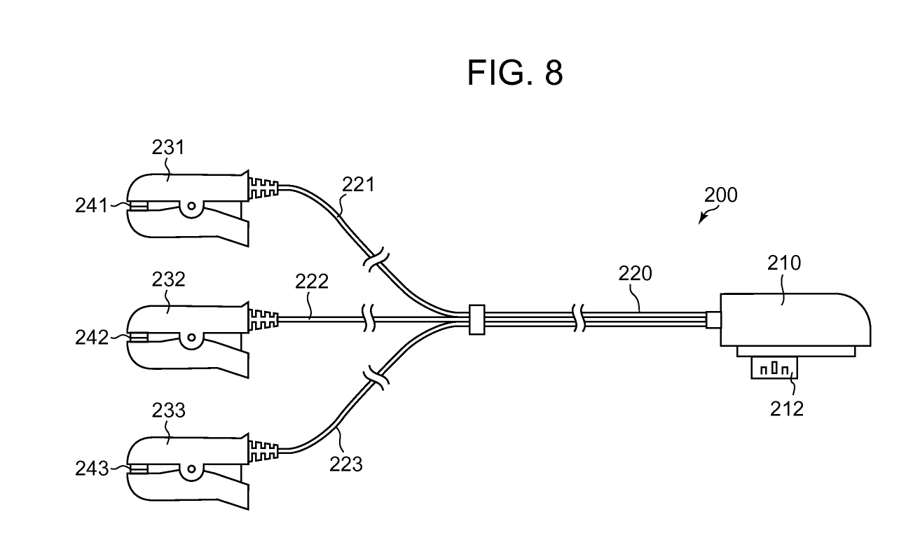

A power source button 141 is disposed in a predetermined position of a top face 133 of the electrocardiograph body 100. The power source button 141 is included in the operating part 140 (see FIG. 12) for operating ON/OFF of the electrocardiograph body 100. Two access covers 130A and 1308 are provided on a predetermined position on the top face 103 of the electrocardiograph body 100. The access cover 130A is provided on the surface of the electrocardiograph body 100 corresponding to the portions of the female connector 131 (see FIG. 10) to which the after-mentioned male connector 210 (see FIG. 8 or FIG. 10) of the lead part 200 for connecting external electrodes is connected and the female connector 132 to which the male connector 410 (see FIG. 10) of the lead part 400 for connecting external terminals is connected. The access cover 130A is configured to cover these female connectors when it is closed and to expose them when it is opened. On the other hand, the access cover 1308 is provided on the surface of the electrocardiograph body 100 corresponding to the portion of the slot where the after-mentioned external memory 134 is inserted, to cover the slot when it is closed and expose it when it is opened.

As shown in FIGS. 2 and 5, various operating buttons are located at a predetermined position on the bottom face 104 of the electrocardiograph body 100. Specifically, menu button 143, decision button 144, left scroll button 145, and right scroll button 146 are disposed on the bottom face 104 of the electrocardiograph body 100. These operating buttons are included in the operating part 140 (see FIG. 12). The menu button 143 is an operating button for entering an instruction to retrieve the monitored results stored in the electrocardiograph body 100, and the decision button 144 is an operating button to perform respective operations. The left scroll button 145 and right scroll button 146 are operating buttons for scrolling and displaying charts as monitored results and guide information, etc. in the display part 148.

The access cover 130C is provided on the predetermined portion on the bottom face 104 of the electrocardiograph body 100. The access cover 130C is provided corresponding to the portion of a battery containing part for containing the after-mentioned battery 160 (see FIG. 12) to cover the battery containing part when it is closed and expose it when it is opened.

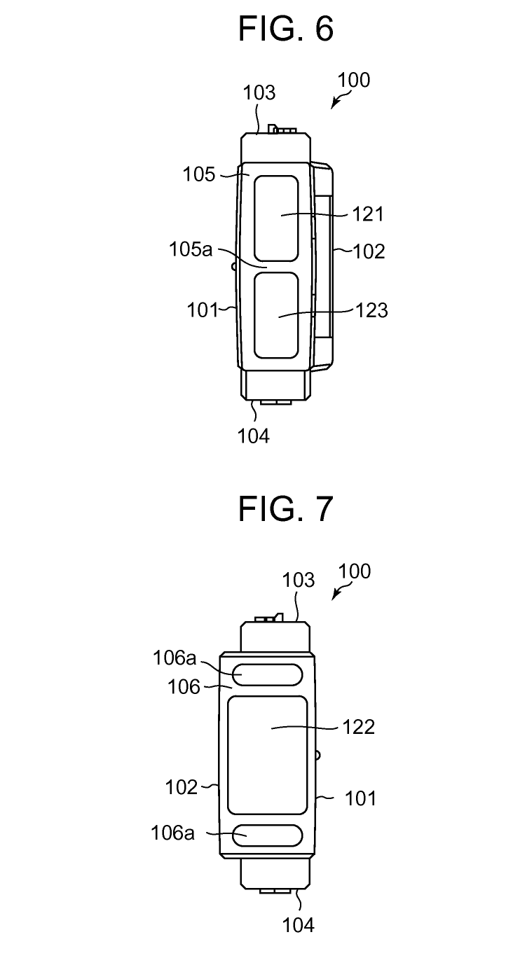

As shown in FIGS. 1 and 6, negative electrode 121, being one of the exposed electrodes, and indifferent electrode 123 also being one of the exposed electrodes, for introducing a reference electrical potential with respect to a potential variation of a body are located on a right lateral face 105 located at one end of the electrocardiograph body 100 in a longitudinal direction. The right lateral face 105 is configured to have a contoured surface to fit with an index finger of the right hand of a subject when the subject takes a posture for monitoring the electrocardiographic waveform based on the after-mentioned first monitoring mode. The right lateral face 105 has a concave portion 105a elongated vertically to accept the index finger of the right hand of the subject.

The above-mentioned negative electrode 121 and indifferent electrode 123 are made of an electrically conductive material. Further, the negative electrode 121 and indifferent electrode 123 are configured such that their surfaces are exposed on the outer surface of the electrocardiograph body 100 in the concave portion 105a provided on the right lateral face 105. The negative electrode 121 is positioned near the top face 103 on the right lateral face 105, while the indifferent electrode 123 is positioned near the bottom face 104 on the right lateral face 105.

As shown in FIGS. 2 and 7, the positive electrode 122 is located as one of exposed electrodes on the left lateral face 106 located at the other end of the electrocardiograph body 100 in a longitudinal direction. The positive electrode 122 is made of an electrically conductive material and formed to protrude a little from the left lateral face 106. Further, a pair of convex portions 106a is provided on the left lateral surface 106. The positive electrode 122 is located in between the pair of convex portions on the left lateral surface 106. The convex portions 106a are to improve contact stability between the positive electrode 122 and the body surface when the left lateral surface 106 of the electrocardiograph body 100 is applied onto a body surface of the subject.

FIG. 8 is a frame format showing the structure of the lead part for connecting an external electrode contained in the portable electrocardiograph set according to the first embodiment of the invention. With reference to FIG. 8, the structure of the lead part for connecting an external electrode contained in the portable electrocardiograph set according to the first embodiment of the invention is shown.

As shown in FIG. 8, the lead part 200 included in portable electrocardiograph set 1A according to the first embodiment is configured separately from the electrocardiograph body 100. The lead part 200 is constituted principally by the male connector 210 detachably attached to the electrocardiograph body 100, long cables 221 to 223 extending from the male connector 210, and clips 241 to 243 attached to the ends of the long cables 221 to 223 respectively.

The male connector 210 has a connection terminal 212 which is to be detachably connected to the connection terminal of the female connector 131 in the above-mentioned electrocardiograph body 100. The cables 221 to 223 are bundled to make a cable assembly at the end near to the male connector 210 and are separated at the end near to the clips 231 to 233 respectively. The clips 231 to 233 are formed as crocodile clip connectors having contacts 241 to 243 at the tips respectively.

The contacts 241 to 243 of the clips 231 to 233 are configured to contact the corresponding contacts of the external electrodes such that the contacts provided on the external electrodes are electrically connected to the cables 221 and 223 when monitoring the electrocardiographic waveform according to the selected after-mentioned second embodiment. In the portable electrocardiograph set 1A adopting the three-point induction method, three cables with clips constitute the lead part 200 for connecting external electrodes corresponding to the three electrodes, i.e. positive electrode, negative electrode and indifferent electrode.

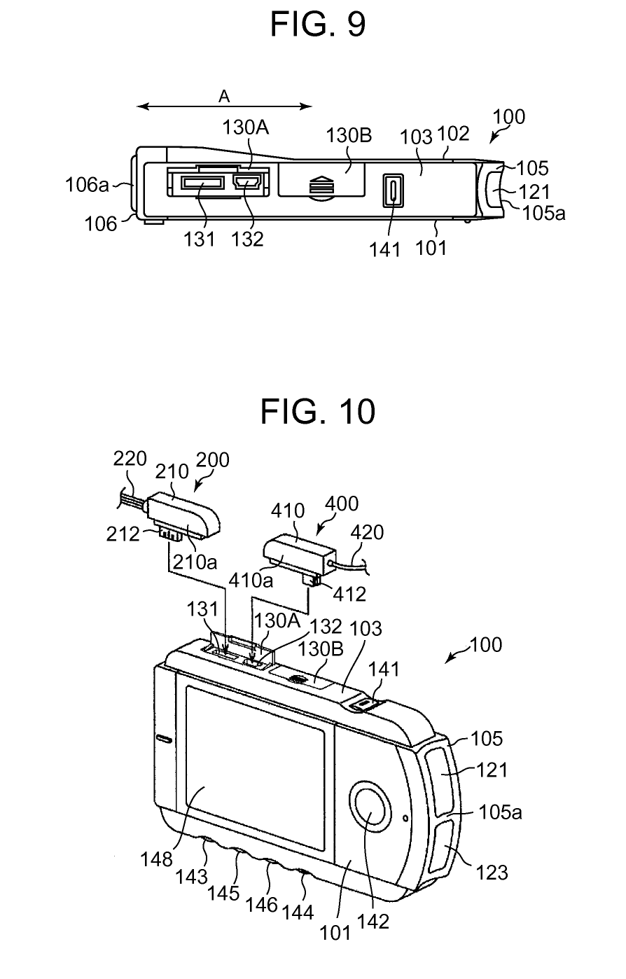

FIG. 9 is a top view of the electrocardiograph body keeping open an access cover for inserting either the lead part for connecting external electrodes or the lead part for connecting external terminals. FIG. 10 is a frame format showing the manner that either the lead part for connecting external electrodes or the lead part for connecting external terminals is inserted into the electrocardiograph body.

As shown in FIGS. 9 and 10, when the access cover 130A provided on the top face 103 of the electrocardiograph body 100 is open, the female connector 131 which is connectable to the male connector 210 of the lead part 200 for connecting external electrodes and the female connector 132 which is connectable to the male connector 410 of the lead part 400 for connecting external terminals are exposed. The female connectors 131 and 132 are juxtaposed to each other in the longitudinal direction of the electrocardiograph body 100. The male connector 210 of the lead part 200 for connecting external electrodes is connected to the female connector 131 when monitoring the electrocardiographic waveform according to the after-mentioned second embodiment, while the male connector 410 of the lead part 400 is connected to the female connector 132 particularly when the electrocardiographic waveform stored in the electrocardiograph body 100 are transferred to an external terminal, such as a PC (see FIG. 12).

When the male connector 210 of the lead part 200 is connected to the female connector 131, the connection terminal 212 of the male connector 210 is inserted in the connection terminal of the female connector 131 and the cover 210a of the male connector 210 blocks the connection terminal of the female connector 132, thus the male connector 410 of the lead part 400 for connecting external terminals cannot be connected to the female connector 132.

Similarly, when the male connector 410 of the lead part 400 is connected to the female connector 132, the connection terminal 412 of the male connector 410 is inserted in the connection terminal of the female connector 132 and the cover 410a of the male connector 410 blocks the connection terminal of the female connector 131, thus the male connector 210 of the lead part 200 for connecting external electrodes cannot be connected to the female connector 131.

In this way, the male connector 210 of the lead part 200 and the male connector 410 of the lead part 400 are alternatively connected to the electrocardiograph body 100.

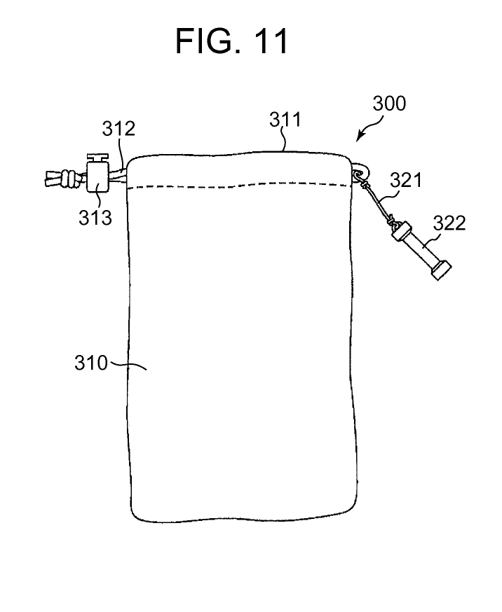

FIG. 11 is a front view showing the structure of a drawstring bag included as a container member in the portable electrocardiograph set according to the first embodiment. The structure of a drawstring bag included as container member in the portable electrocardiograph set according to the first embodiment is described with reference to FIG. 11.

As shown in FIG. 11, the drawstring bag 300 included in the portable electrocardiograph set 1A as container member includes the bag portion 310, the string 312 and the lock member 313. The bag portion 310 has a containing space inside capable of containing such as the above-mentioned electrocardiograph body 100 and the lead part 200 for connecting external electrodes. The opening 311 for putting in or taking out the electrocardiograph body 100 and the lead part 200 for connecting external electrodes is provided at the upper end of the bag portion 310. The above-mentioned string 312 and the lock member 313 are used to open or close the opening 311 which can be opened or closed by operating the lock member 313 attached to the string 312.

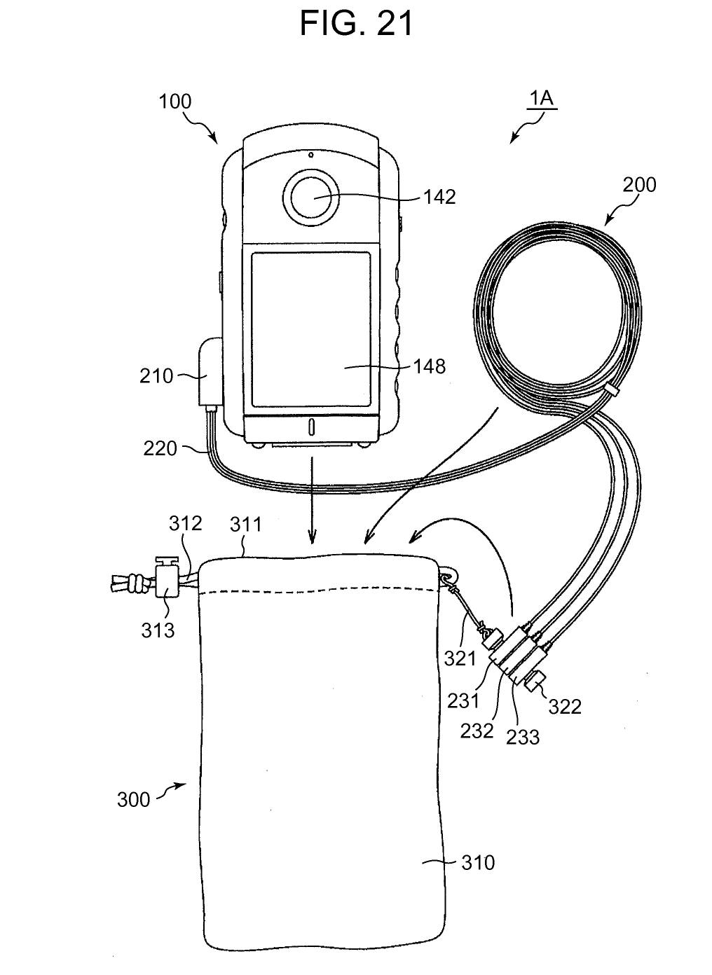

The drawstring bag 300 is provided with a clip fixing member 322 through the strap 321. The clip fixing member 322 is made up of a member with an appropriate rigidity, having a shape such that the clips 231 to 233 of the lead part 200 for connecting external electrodes can be fixed thereto. Specifically, the clip fixing member 322 is formed with a member in the shape of rod and configured such that the clips 231 and 233 can be fixed thereto side-by-side (see FIG. 21). The clip fixing member 322 is provided near the opening 311 and can be used both when it is inside and when it is outside of the bag portion 310.

The portable electrocardiograph set 1A according to the first embodiment is provided with at least the above-mentioned electrocardiograph body 100, the lead part 200 for connecting external electrodes, and the drawstring bag 300 as container member. It is also provided with the above-mentioned lead part 400 for connecting external terminals, the external electrodes (negative electrode 451, positive electrode 452 and indifferent electrode 453 (see FIGS. 12, 19 and 20)), and external memory 134 (see FIG. 12), etc. These items are described later.

FIG. 12 is a function block diagram of the electrocardiograph body, etc. included in the portable electrocardiograph set according to the first embodiment. The function block diagram of the electrocardiograph body, etc. included in the portable electrocardiograph set according to the first embodiment is described with reference to FIG. 12.

As shown in FIG. 12, the electrocardiograph body 100 principally includes the negative electrode 121, positive electrode 122 and indifferent electrode 123, being the exposed electrodes, the operating part 140 and the processing circuit 150. The operating part 140 is constituted by the above-mentioned power source button 141, monitor button 142, menu button 143, decision button 144, left scroll button 145 and right scroll button 146. The electrocardiograph body 100 is connected to the negative electrode 451, positive electrode 452, and indifferent electrode 453 as the external electrodes through the lead part 200 for connecting external electrodes as necessary. The connection has already been described using FIGS. 8 and 10.

The processing circuit houses the electrocardiogram monitoring circuit for processing the biological electrical signals detected by the exposed electrodes or the external electrodes to monitor them as the electrocardiographic waveform. Specifically, the processing circuit 150 includes the amplifier circuit 151 for amplifying the biological electrical signals detected by the exposed electrodes or the external electrodes, the filter circuit 152 for eliminating the noise content from the signals outputted from the amplifier circuit 151, the A/D converter 153 for converting analog signals to digital signals, the CPU 154 (Central Processing Unit), the internal memory 155 having the ROM and RAM, and the timer 156 for outputting time data to the CPU 154. The amplifying circuit 151 differentially amplifies the output voltage signals (biological electrical signals) of the negative electrode 121 and the positive electrode 122 with reference to the output voltage signal of the indifferent electrode 123 (or the output voltage signal (biological electrical signals) of the negative electrode 451 and the positive electrode 452 with reference to the output voltage signal of the indifferent electrode 453). Further, the filter circuit 152 may adopt a band pass filter having the pass band of, for example 0.5 Hz to 35 Hz.

The processing circuit 150 is connected to the above-mentioned exposed electrodes (external electrodes as necessary), operating part 140, display part 148, and power source 160. Further, when the external memory 134 is inserted in the slot, the external memory is also connected to the processing circuit 150. Furthermore, the external terminal 133 is also connected to the processing circuit 150 through the lead part 400 for connecting external terminals.

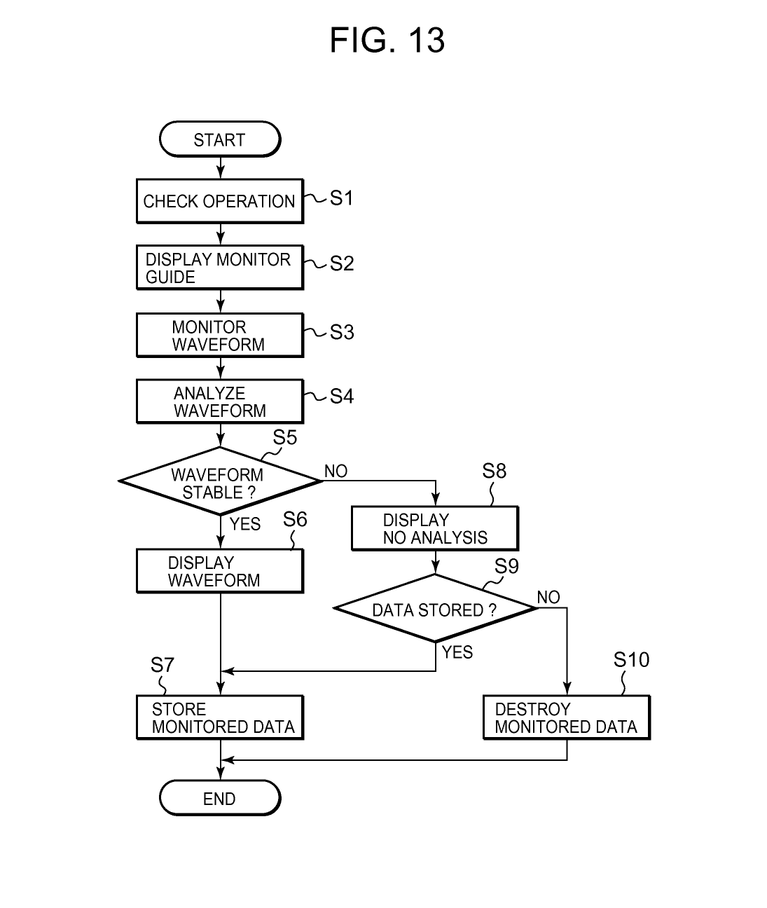

The CPU 154 performs the analysis of the digital signal inputted from A/D converter 153, and receives the instruction signal from various operating buttons included in the operating part 140 to perform processing corresponding to the received instruction signal. Further, the CPU performs writing and reading to and from the internal memory 155 and display controlling for the display part 148. FIG. 13 is a flowchart of the electrocardiograph body included in the portable electrocardiograph set according to the first embodiment. Hereinafter, the flowchart of the electrocardiograph body included in the portable electrocardiograph set according to the first embodiment is described with reference to FIG. 13.

The steps of the flowchart shown in FIG. 13 are preliminarily stored as a program in the ROM of the inner memory 155 and the CPU 154 reads the program and prosecutes the same to perform the above-mentioned steps.

As shown in FIG. 13, when the subject presses the power source button 141 to power on the electrocardiograph body 100, an operation check is performed (step S1), then a monitor guide appears on the display 148 (step S2). For example, a guide to the posture the subject is supposed to take when monitoring the electrocardiographic waveform is displayed. The posture for monitoring the electrocardiographic waveform is described later.

Subsequently, when the subject presses the monitor button 142 while taking a predetermined monitoring posture, the monitor and analysis of the electrocardiographic waveform are performed (step S3, S4). The electrocardiographic waveform converted to the digital signal by the A/D converter 153 is temporally stored in the RAM of the inner memory 155.

The analysis of the electrocardiographic waveform is to detect characteristics of waveforms showing arrhythmia, ischemic myocardium, etc., characteristics of cycles showing bradycardia, tachycardia, etc., or waveforms which cannot be analyzed due to noise or base line fluctuation and analyze the detected results. The monitoring and analyzing of the electrocardiographic waveform is performed in accordance with well-known methods.

As a result of the analysis of the waveform, it is judged whether the obtained electrocardiographic waveform is stable (step S5). If it is judged that the waveform is stable (Yes in step S5), then the program goes to step S6 and the display 148 displays messages based on the monitored waveform, cardiac rate and analysis results of the waveform. In step S6, the CPU 154 makes a message from the analysis results of the electrocardiographic waveform and displays it on the display 148. The cardiac rate per time unit is also displayed with the message. The cardiac rate can be obtained by a well-known proceeding based on the electrocardiographic waveform. The program goes to step S7 after completing processing of step S6. On the other hand, if it is judged that the electrocardiographic waveform is not stable (No in step S5), the program goes to step S8 and the computer 154 displays that the analysis of the electrocardiographic waveform was not performed. Further, the computer 154 displays whether the monitored data should be stored (step S9).

In step S9, it is judged whether the subject selects storing the monitored data corresponding to the message that the analysis of the electrocardiographic waveform was not performed. If storing of the monitored data is selected (Yes in step S9), then the program goes to step S7. If destroying of the monitored data is selected (No in step S9), then the program goes to step S10.

In step S7, the CPU 154 performs processing of storing the monitored data. Specifically, the CPU 154 stores the present time data inputted from the timer 156 and the corresponding electrocardiographic waveform and the analysis results temporarily stored in the RAM in a predetermined area of the inner memory 155. In step S10, the computer 154 destroys the monitored data.

A series of processing as described above are performed in the electrocardiograph body 100 to monitor and store the electrocardiographic waveform. The electrocardiograph body 100 included in the portable electrocardiograph set 1A has a function of retrieving and displaying on the display 148 the monitored data stored in step S7.

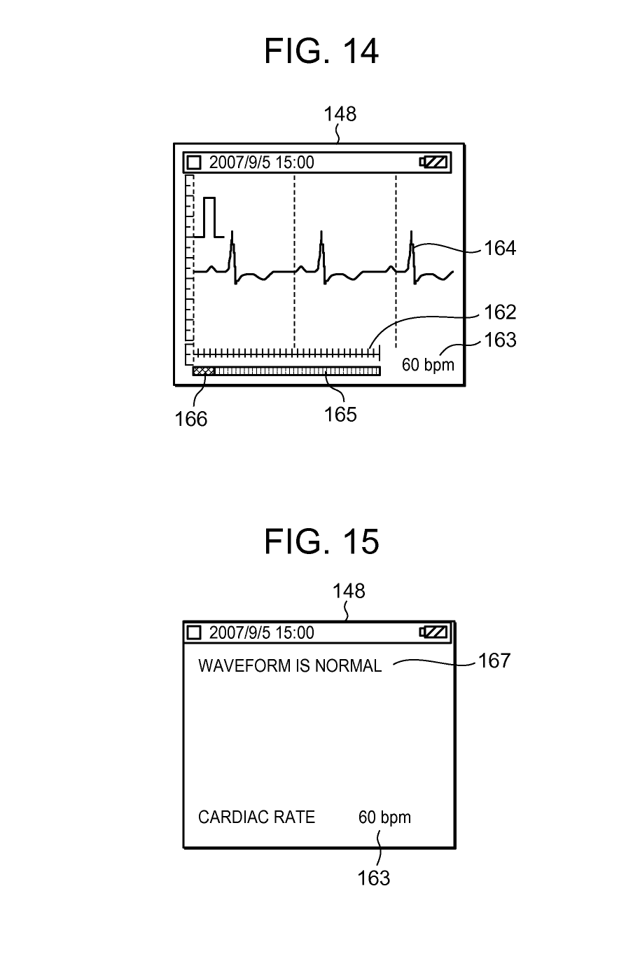

FIGS. 14 and 15 are display examples of the monitored results on the display of the electrocardiograph body included in the portable electrocardiograph set according to the first embodiment. Hereinafter, the display examples are described with reference to FIGS. 14 and 15.

As shown in FIG. 14, a reduced-size waveform 162 of entire wave form throughout a monitoring period is displayed on the lower part of the screen of the display 148 at a stat of displaying, while an enlarged-size waveform 164 for example, for two seconds from the start of displaying is displayed on the upper part of the screen. A scale bar 165 is provided adjacently under the display of the reduced-size waveform 162, displaying a length of monitoring. A pointer 166 is displayed on the scale bar 165 to indicate where the enlarged-size waveform 164 is located in the entire monitoring period. In order to display an enlarged-size waveform 164 during a different monitoring period, the above-mentioned left scroll button 145 and right scroll button 146 are operated to move the pointer 166 on the scale bar 165 to the location corresponding the monitoring period. In this way, the enlarged waveform 164 can be displayed during any two seconds throughout the monitoring period. Further, a beat rate 163 is displayed at a predetermined position on the display 148.

If the subject presses the decision button 144 after recognizing the electrocardiographic waveform on the screen shown in FIG. 14, the CPU retrieves the analysis results corresponding to the waveform shown on the screen from the inner memory 155, make a message and displays it on the screen of the display 148, for example as shown in FIG. 15.

The electrocardiograph body 100 in the portable electrocardiograph set 1A according to the present embodiment has exposed electrodes (negative electrode 121, positive electrode 122 and indifferent electrode 123) on its surface, while it is configured to be connected to the external electrodes (negative electrode 451, positive electrode 452 and indifferent electrode 453) through the lead part 200 for connecting external electrodes. Accordingly, it is possible to monitor the electrocardiogram in two ways, a first monitoring mode using the exposed electrodes and a second monitoring mode using the external electrodes in the portable electrocardiograph set 1A according to the present embodiment. Hereinafter, these two monitoring modes are detailed.

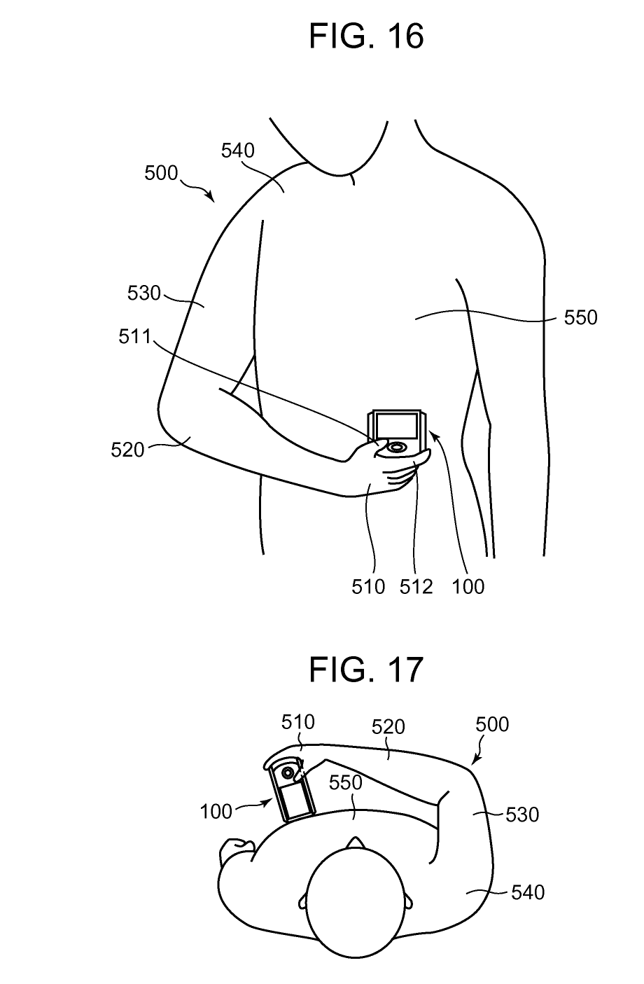

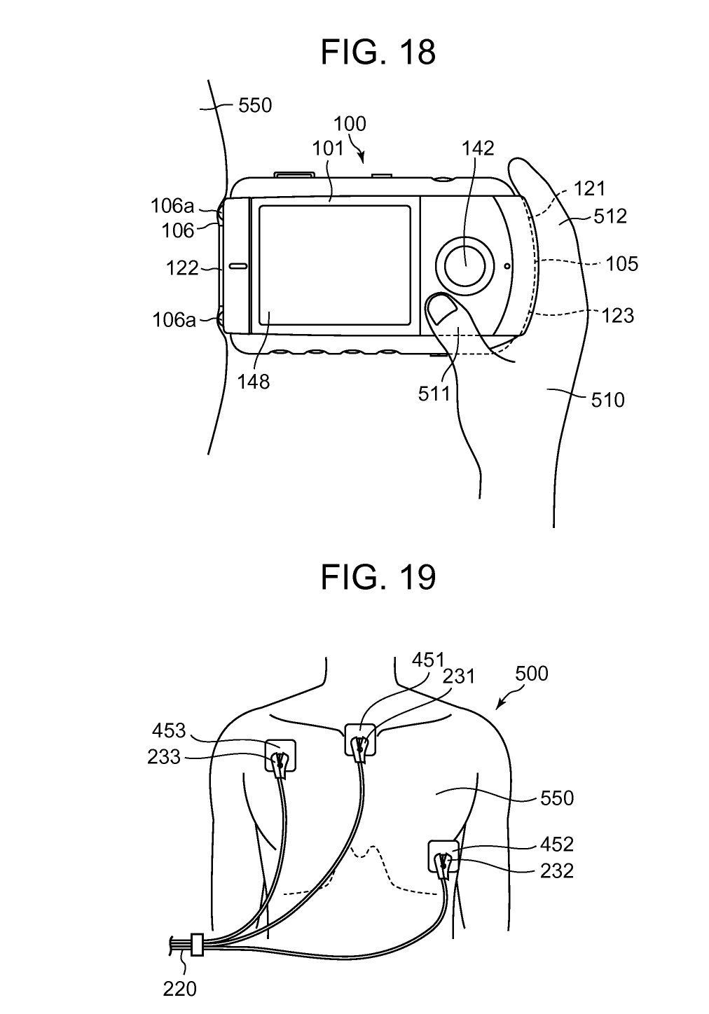

FIGS. 16 and 17 are views showing a posture a subject should take when monitoring an electrocardiographic waveform in the first monitoring mode in the portable electrocardiograph set according to the first embodiment. FIG. 18 is a view showing the right hand of a subject holding an electrocardiograph body when the subject takes the postures shown in FIGS. 16 and 17. First, the posture the subject should to take and the operation of the electrocardiograph body 100 when monitoring the electrocardiographic waveform in the first monitoring mode is described with reference to FIGS. 16 to 18. The lead part 200 for connecting external electrodes is not used when monitoring the electrocardiographic waveform in the first monitoring mode.

As shown in FIGS. 16 and 17, the subject 500 directly contacts the positive electrode 122 provided at one end of the electrocardiograph body 100 (near the left lateral face 106 of the electrocardiograph body 100) to the skin over the fifth intercostal space (between ribs 5 & 6) in the anterior axillary line located in the lower left side of the breast 550, while holding the other end of the electrocardiograph body 100 (near the right lateral face 105 of the electrocardiograph body 100) in the right hand 510. The subject 500 presses the monitor button 142 provided on the front face 101 of the electrocardiograph body 100 with the thumb 511 of the right hand 510. After pressing the monitor button 142, the subject 500 keeps on contacting the above-mentioned one end of the electrocardiograph body 100 to the above-mentioned position of the breast 550 while holding the above-mentioned other end of the electrocardiograph body 100 in the right hand until monitoring of the electrocardiographic waveform is terminated.

As shown in FIG. 18, the subject 500 holds the electrocardiograph body 100 with the right hand 510 such that the front face 101 of the electrocardiograph body 100 faces upwards, while applying the index finger 512 on the right lateral face 105 and grasping the electrocardiograph body 100 with the thumb and the middle finger applied to both front and back faces 101, 102 respectively. The index finger 512 of the right hand 510 is applied to the contour of the right lateral face 105 to fit in the concave portion 105a and contact the negative electrode 121 and indifferent electrode 123 provided on the concave portion 105a.

By taking the above-mentioned posture, the negative and indifferent electrodes located on the right lateral face 102 of the electrocardiograph body 100 contact the index finger 512 of the subject's right hand 510 and the positive electrode 122 located on the left lateral face 106 of the electrocardiograph body 100 contacts the breast 550 of the subject 500. In this way, an electrical circuit for monitoring the electrocardiographic waveform is formed in the subject's body through the right hand 510 contacting the negative electrode 121 and indifferent electrode 122, the lower arm 520 non-contacting the breast 550, the upper arm 530 non-contacting the breast 550 either, right shoulder 540 and the breast 550 on which the positive electrode 122 is pressed.

Accordingly, monitoring of the electrocardiographic waveform is enabled by maintaining the above-mentioned monitoring posture. FIGS. 16 and 17 show monitoring in a standing-up posture, however monitoring in a recumbent posture or a seated posture are available as well.

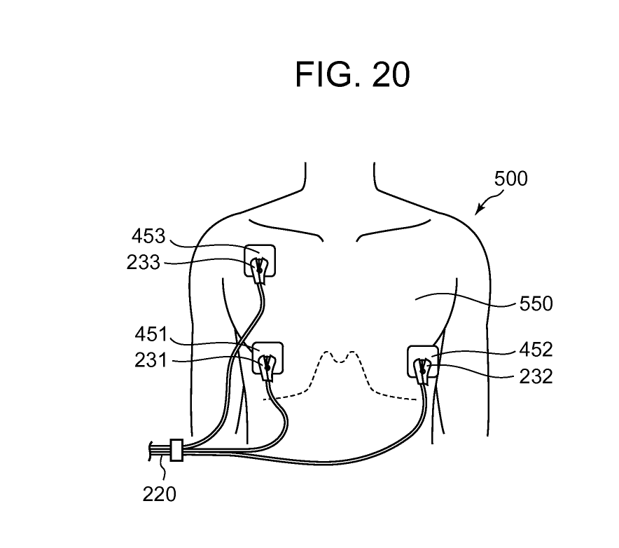

FIGS. 19 and 20 are views showing examples of locations where the external electrodes should be attached when monitoring the electrocardiographic waveform by the second embodiment in the portable electrocardiograph set according to the first embodiment. FIG. 19 shows the locations where the external electrodes should be attached based on the CM5-induction and FIG. 20 shows the locations where the external electrodes should be attached based on the CC5-induction as examples of the second monitoring mode. These CM5 and CC5 inductions are similar to the V5-induction in the 12-induction method (chest leads).

Next, the posture that the subject should take, the locations where the external electrodes should be attached and the operations of the electrocardiograph body 100 including the lead part for connecting external electrodes are described with reference to FIGS. 19 and 20 when monitoring the electrocardiographic waveform based on the second monitoring mode.

First, the male connector 210 of the lead part 200 for connecting external electrodes is inserted into the female connector 131 for connecting external electrodes after opening the access cover 130A of the electrocardiograph body 100 when monitoring the electrocardiographic waveform based on the second monitoring mode. The negative electrode 451, positive electrode 452 and indifferent electrode 453 are respectively attached to predetermined positions of the breast 550 of the suspect 500 as external electrodes as shown in FIGS. 19 and 20. The negative electrode 451, positive electrode 452 and indifferent electrode 453 are made up of disposal and adhesive electrodes with protruded contacts formed to be electrically conducted to the electrodes on the side opposite to the surfaces of the respective electrodes.

Specifically, the negative electrode 451 is attached on the skin of upper breast bone of the patient's breast 550, the positive electrode 452 is attached on the skin over the left fifth intercostal space (between ribs 5 & 6) in the anterior axillary line, and the indifferent electrode 453 is attached on the skin under the right collarbone respectively as shown in FIG. 19 when monitoring the electrocardiographic waveform based on the CM5 induction.

When monitoring the electrocardiographic waveform based on the CC5 induction, the negative electrode 451 is attached on the skin over the right fifth intercostal space (between ribs 5 & 6) in the anterior axillary line of the patient's breast 550, the positive electrode 452 is attached on the skin over the left fifth intercostal space (between ribs 5 & 6) in the anterior axillary line and the indifferent electrode 453 is attached on the skin under the right collarbone respectively as shown in FIG. 20.

Next, the clips 231 to 233 of the lead part 200 are correspondingly fixed to the above-mentioned protruded contacts provided for the negative electrode 451, positive electrode 452 and indifferent electrode 453 respectively. Upon pressing the monitor button 142 of the electrocardiograph body 100, while maintaining the above-mentioned state, monitoring of the electrocardiographic waveform is performed. It is preferable for the subject to take a supine posture during monitoring, however a recumbent posture or seated posture is available as well.

FIG. 21 is a view showing a manner that the electrocardiograph body and the lead part for connecting external electrodes are contained in the drawstring bag as container member in the portable electrocardiograph set according to the first embodiment.

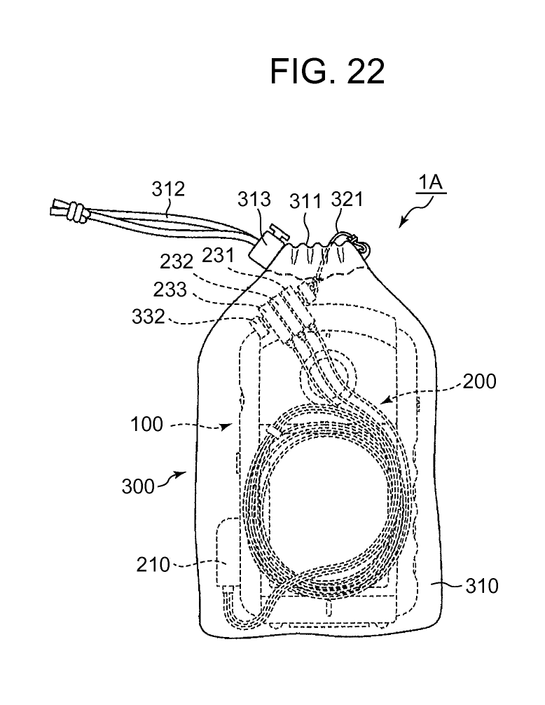

FIG. 22 is a view showing a state where the electrocardiograph body and the lead part for connecting external electrodes are contained in the drawstring bag as container member in the portable electrocardiograph set according to the first embodiment.

Next, the manner that the electrocardiograph body and the lead part for connecting external electrodes are contained and the state where they are contained in the drawstring bag are described with reference to FIGS. 21 and 22.

The portable electrocardiograph set 1A is provided with the electrocardiograph body 100, the lead part 200 for connecting external electrodes, and the drawstring bag 300 as container member as described above. When containing and carrying the electrocardiograph body 100 and the lead part 200 for connecting external electrodes in the drawstring bag 300, the male connector 210 of the lead part 200 is connected to the female connector 131 of the electrocardiograph body 100 while all the clips 231 to 233 included in the lead part 200 are fixed to the clip fixing member 322 that is attached to the drawstring bag 300 as shown in FIG. 21. The cables 221 to 223 of the lead part 200 are bundled in a loop shape and contained in the drawstring bag 300 together with the electrocardiograph body 100 and the clip fixing member 322 having all the clips 231 to 233 fixed thereto while the cables 221 to 223 are maintained in a loop shape. Then, the opening of the drawstring bag 300 is squeezed closed using the string 312 and lock member 313 as shown in FIG. 22.

In this way, the electrocardiograph body 100 and the lead part 200 for connecting external electrodes are portably contained in the drawstring bag 300. The external electrodes (negative electrode 451, positive electrode 352 and indifferent electrode 453), lead part for connecting external terminals 400, and external memory 132, etc. may be contained in the drawstring bag 300 together with the electrocardiograph body 100 and the lead part 200 for connecting external electrodes as necessary. Since the portable electrocardiograph set 1A is capable of containing the electrocardiograph body 100 and the lead part 200 for connecting external electrodes in the drawstring bag 300 such that all the clips 231 to 233 included in the lead part t 200 are fixed to the clip fixing member 322 provided at the drawstring bag 300, the cables 221 to 223 included in the lead part 200 are prevented from getting entangled with each other. Therefore, the portable electrocardiograph set 1A may be carried without having the cables 221 to 223 entangled, thereby enabling monitoring of the electrocardiographic waveform promptly without inconvenience.

The clip fixing member 322 attached to the drawstring bag 300 is preferably made of a conductive material in the present embodiment. At least the surface of a rod forming the clip fixing member 322 where the clips 231 to 233 are fixed should be made of the conductive material. Breakage of the wire may be easily checked for the lead part 200 according to the above-mentioned structure. Hereinafter, the method of checking wire breakage is described.

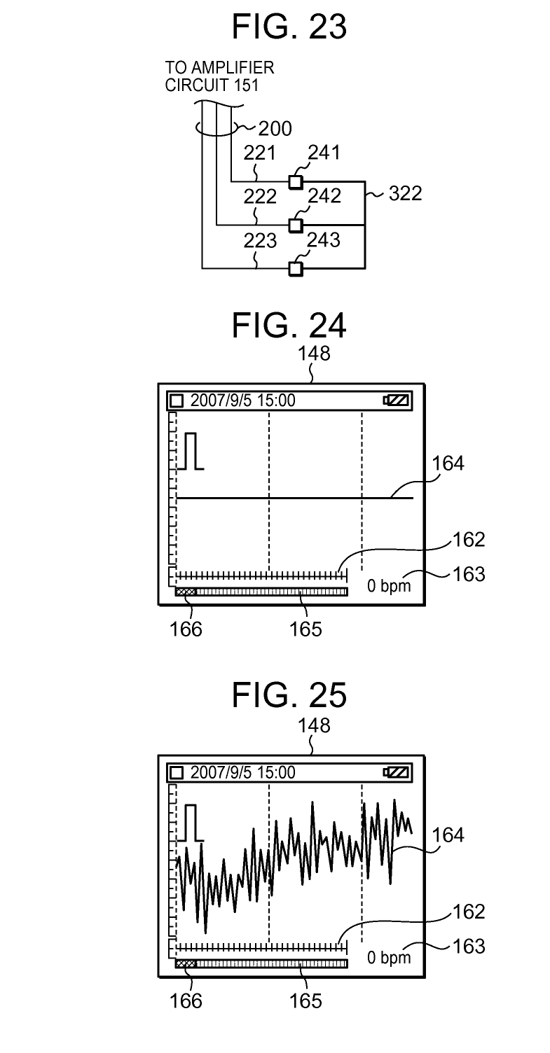

FIG. 23 is a frame format showing an electrical connection when the check of wire breakage is conducted in the portable electrocardiograph set according to the first embodiment.

When the clips 231 to 233 of the lead part 200 for connecting external wires are fixed to the surface of the clip fixing member 322 made of the electrically conductive member as shown in FIG. 23, the contacts 241 to 243 provided on the respective clips 231 to 233 are electrically connected through the clip fixing member 322. The contacts 241 to 241 of the clips 231 to 233 are electrically connected to the electrocardiogram monitoring circuit provided in the electrocardiograph body 100 through the respective cables 221 to 223.

Accordingly, upon pressing the monitor button 142 of the electrocardiograph body 100 while the clips 231 to 233 of the lead part 200 are fixed to the clip fixing member 322, breakage of the wire may be checked for the wires 221 to 223 by recognizing the waveform displayed on the display 148 of the electrocardiograph body 100. FIG. 24 is a view showing a display example when no wire breakage occurs in the cables, while FIG. 25 is a view showing a display example when a wire breakage occurs in the cables.

Since no open portion exists in the monitoring circuit unless wire breakage occurs, no difference of potential is monitored and the waveform 164 becomes stable with a potential difference of zero as shown in FIG. 24. On the contrary, if a wire breakage occurs in any of cables 221 to 223, an open portion exists in the monitoring circuit, causing unstable waveforms 164 to be monitored as shown in FIG. 25. In this way, wire breakage may be checked for cables 221 to 223 by pressing the monitor button 142 of the electrocardiograph body 100 while having the clips 231 to 233 fixed to the clip fixing member 322.

Fixing the clips 231 to 233 to the clip fixing member 322 is the same as the state of containing the electrocardiograph body 100 and lead part 200 in the drawstring bag 300, thus if the electrocardiograph body 100 and lead part 200 are stored in the drawstring bag 300 with all the clips 231 to 233 being fixed to the clip fixing member 322, wire breakage may be immediately checked for the cables 221 to 223 only by taking the electrocardiograph body 100 out of the drawstring bag 300. Therefore, the portable electrocardiograph set 1A may be configured to carry the cables 221 to 223 without getting entangled and easily check wire breakage for the cables 221 to 223, thus enabling a prompt monitoring of the electrocardiographic waveform without inconvenience and fail.

### Second Embodiment

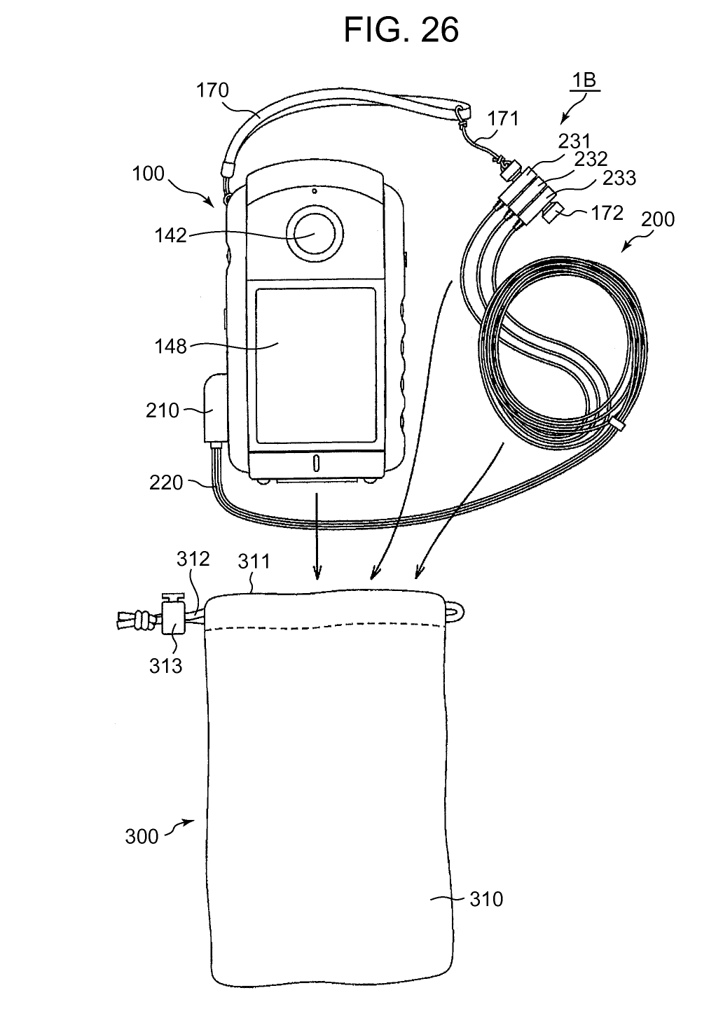

FIG. 26 is a view showing a manner that the electrocardiograph body and the lead part for connecting external electrodes are contained in the drawstring bag as container member in the portable electrocardiograph set according to the second embodiment.

The portable electrocardiograph set 1B has the same configuration as the portable electrocardiograph set 1A except for the location where the clip fixing member is fixed, and includes the electrocardiograph body 100, the lead part 200 for connecting external electrodes and drawstring bag 300 as container member as the portable electrocardiograph set 1A does. Accordingly, the same symbols are used for the same components and corresponding portions, not repeating the same descriptions.

As shown in FIG. 26, the clip fixing member 172 is attached through strap 171 to a hand strap 170 that is attached to the electrocardiograph body 100 in the portable electrocardiograph set 1B.

When the electrocardiograph body 100 and the lead part 200 are contained in the drawstring bag 300 to carry, the male connector 210 of the lead part 200 is connected to the female connector 131 of the electrocardiograph body 100 and all the clips 231 to 233 included in the lead part 200 are fixed to the clip fixing member 172 that is attached to the electrocardiograph body 100 as shown in FIG. 26. Then, the cables 221 to 223 of the lead part 200 are bundled in a loop and put in the bag portion 310 of the drawstring bag 300 together with the electrocardiograph body 100 and the clip fixing member 172 having clips 231 to 233 fixed thereto with the loop shape being maintained. And, the opening 311 of the drawstring bag 300 is squeezed and closed using the string 312 and lock member 313. Thus, the electrocardiograph body 100 and the lead part 200 are contained in the drawstring bag 300 such that they may be conveniently carried.

Since the portable electrocardiograph set 1B is capable of containing the electrocardiograph body 100 and the lead part 200 for connecting external electrodes in the drawstring bag 300 such that all the clips 231 to 233 included in the lead part t 200 are fixed to the clip fixing member 172 provided at the electrocardiograph body 100, the cables 221 to 223 included in the lead part 200 are prevented from getting entangled with each other. Therefore, the portable electrocardiograph set 1B may be carried without having the cables 221 to 223 entangled, thereby enabling to monitor the electrocardiographic waveform promptly without inconvenience.

Breakage of the cables 221 to 223 may be checked as well in the same manner as the portable electrocardiograph set 1A by forming the surface that the clips 231 to 233 contact in the clip fixing member 172 with an electrically conductive material in the portable electrocardiograph set 1B according to the second embodiment.

### Third Embodiment

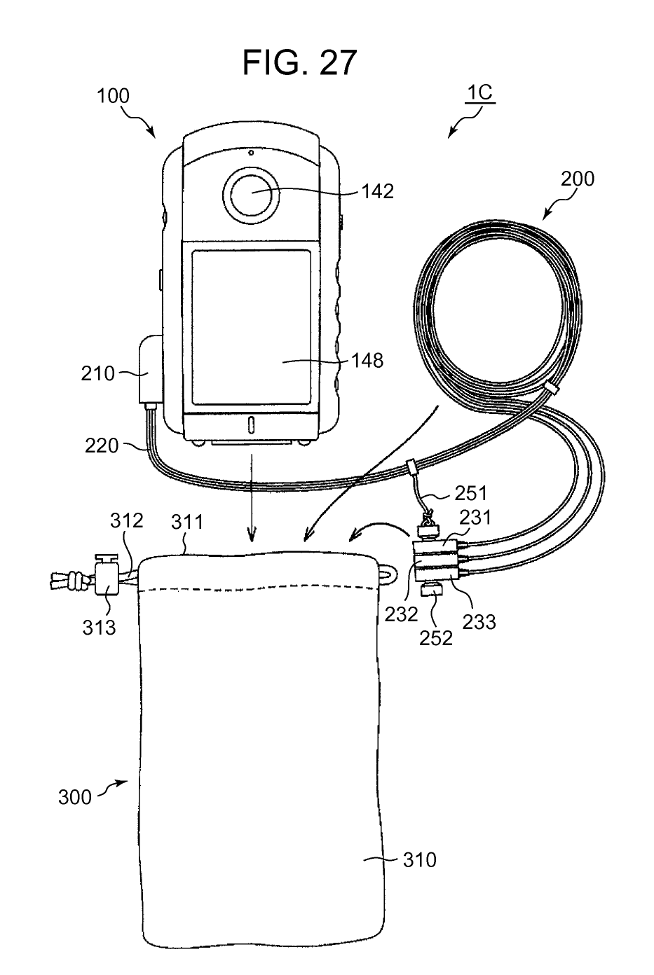

FIG. 27 is a view showing a manner that the electrocardiograph body and the lead part for connecting external electrodes are contained in the drawstring bag as container member in the portable electrocardiograph set according to the third embodiment.

The portable electrocardiograph set 10 has the same configuration as the portable electrocardiograph set 1A except for the location where the clip fixing member is fixed, and includes the electrocardiograph body 100, the lead part 200 for connecting external electrodes and drawstring bag 300 as container member as the portable electrocardiograph set 1A does. Accordingly, the same symbols are used for the same components and corresponding portions, not repeating the same descriptions.

The clip fixing member 252 is attached to the assembly cable 220 of the lead part 200 for connecting external electrodes through the strap 251 in the portable electrocardiograph set 10 according to the third embodiment as shown in FIG. 27.

When the electrocardiograph body 100 and the lead part 200 are contained in the drawstring bag 300 for carrying, the male connector 210 of the lead part 200 is connected to the female connector 131 of the electrocardiograph body 100 and all the clips 231 to 233 included in the lead part 200 are fixed to the clip fixing member 252 that is attached to the lead part 200 as shown in FIG. 27. Then, the cables 221 to 223 of the lead part 200 are bundled in a loop and put in the bag portion 310 of the drawstring bag 300 together with the electrocardiograph body 100 and the clip fixing member 252 having clips 231 to 233 fixed thereto with the loop shape being maintained. And, the opening 311 of the drawstring bag 300 is squeezed and closed using the string 312 and lock member 313. Thus, the electrocardiograph body 100 and the lead part 200 are contained in the drawstring bag 300 such that they may be conveniently carried.

Since the portable electrocardiograph set 10 is capable of containing the electrocardiograph body 100 and the lead part 200 for connecting external electrodes in the drawstring bag 300 such that all the clips 231 to 233 included in the lead part t 200 are fixed to the clip fixing member 252 provided at the lead part 200 for connecting external electrodes, the cables 221 to 223 included in the lead part 200 are prevented from getting entangled with each other. Therefore, the portable electrocardiograph set 10 may be carried without having the cables 221 to 223 entangled, thereby enabling a prompt monitoring of the electrocardiographic waveform without inconvenience.

Breakage of the cables 221 to 223 may be checked as well in the same manner as the portable electrocardiograph set 1A by forming the surface of the clip fixing member 252 that the clips 231 to 233 contact with an electrically conductive material in the portable electrocardiograph set 10 according to the third embodiment.

Although the above-mentioned portable electrocardiograph sets 1A, 1B and 10 according to the first to third embodiments are configured to have the clip fixing member attached to either the drawstring bag as containing member, the electrocardiograph body, or the lead part, the clip fixing member may be provided not attached to any of these members. On such occasion, the clip fixing member is contained in the drawstring as containing member together with the lead part with the clips being fixed to the clip fixing member.

Although the above-mentioned portable electrocardiograph sets 1A, 1B and 10 according to the first to third embodiments are configured to monitor the electrocardiographic waveform using both exposed electrodes and external electrodes, the electrocardiograph body may be configured without the exposed electrodes as well. In other words, the electrocardiograph body configured to use only the external electrodes without using the exposed electrodes to monitor the electrocardiographic waveform may be applied in practicing the invention.

Further, although the lead part for connecting external electrodes to the electrocardiograph body are described as being detachably attachable to the electrocardiograph body according to the first to third embodiments, the portable electrocardiograph set having both electrocardiograph body and lead part integrally configured may be applied in practicing the invention.

Further, although the drawstring bag is described as exemplary container member in the first to third embodiments described above, various other containers such as a soft case, hard case, and system bag are available as container member.

Furthermore, although the strap in a string shape is described in the first to third embodiments as a strap for fixing the clip fixing member to the electrocardiograph body, the lead part for connecting external electrodes, or the containing member, the strap in a chain shape or the belt-like strap, etc. may be used. Otherwise, the clip fixing member may be formed integrally with these members without using a strap.

The embodiments as described above are all examples which should not be taken to limit the scope of the invention. The scope of the invention is to be defined not by the above description but by claims and intended to include all equivalents and modifications without departing from the scope of the invention.

1. A portable electrocardiograph set comprising:
an electrocardiograph body including an electrocardiogram monitoring circuit,
a lead part having two or more cables with respective clips for connecting an external electrode to the electrocardiogram monitoring circuit, and
a containing member for containing and carrying the electrocardiograph body and the lead part,
wherein a clip fixing member configured to enable all of the clips included in the two or more cables to be simultaneously fixed thereto is provided at either the containing member, the lead part or the electrocardiograph body.

2. The portable electrocardiograph set according to claim 1, wherein the clip fixing member can be used within and outside of the containing member.

3. The portable electrocardiograph set according to claim 1, wherein the clip fixing member has at least its surface made of an electrically conducting material.

4. The portable electrocardiograph set according to claim 1, wherein the clip fixing member is made of a rigid material.

5. The portable electrocardiograph set according to claim 1, wherein the clip fixing member is attached to either the containing member, the lead part or the electrocardiograph body through an elongated flexible attaching member or a belt-like strap.

6. The portable electrocardiograph set according to claim 1, wherein the lead part is detachably attached to the electrocardiograph body.

7. The portable electrocardiograph set according to claim 1, wherein the electrocardiograph body has on its surface an exposed electrode electrically connected to the electrocardiogram monitoring circuit for monitoring the electrocardiogram.

8. The portable electrocardiograph set according to claim 1, wherein the containing member is a drawstring bag.

9. A portable electrocardiograph set comprising:
an electrocardiograph body including an electrocardiogram monitoring circuit,
a lead part having two or more cables with respective clips for connecting external electrodes to the electrocardiogram monitoring circuit,
a clip fixing member configured to enable all of the clips included in the two or more cables to be simultaneously fixed thereto, and
a containing member for containing and carrying the electrocardiograph body, the lead part and the clip fix member,
wherein the containing member is configured to contain the clip fixing member together with the lead part with the clips being fixed to the clip fixing member.
